(19) United States
(12) Patent Application Publication
(10) Pub. No.: US 2010/0099652 A1
(43) Pub. Date: Apr. 22, 2010

(54) NOVEL COMPOSITION BASED ON CHOLEST-4-EN-3-ONE OXIME

(75) Inventors: Cyrille Drouot, Draguignan (FR); Patrick Berna, Cassis (FR)

Correspondence Address: BROWDY AND NEIMARK, P.L.L.C.;624 NINTH STREET, NW, SUITE 300, WASHINGTON, DC, 20001-5303, US

(73) Assignee: TROPHOS, Marseille Cedex 9 (FR)

(21) Appl. No.: 12/593,441

(22) PCT Filed: Mar. 26, 2008
(86) PCT No.: PCT/FR2008/000406

§ 371 Date: Sep. 28, 2009

(30) Foreign Application Priority Data
Mar. 28, 2007 (FR) 0702247

(51) Int. Cl. A61K 31/56

(52) U.S. Cl. 514/171

(57) ABSTRACT

A subject of the invention is a novel composition, particularly a pharmaceutical composition comprising cholest-4-en-3-one oxime and an oil chosen from sesame oil, olive oil, soya oil, cotton oil, or a mixture of commercial medium-chain triglycerides (ESTASAN®, MYGLIOL®), or a mixture of these oils, preferably sesame oil, olive oil or soya oil, even more preferably sesame oil.

The present application relates to a composition, particularly a pharmaceutical composition comprising at least a compound of formula I

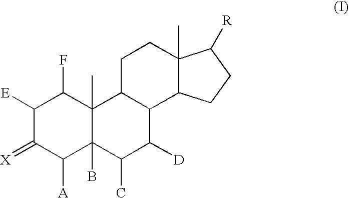

in which X represents an ═N—OH group,
R represents a group chosen from

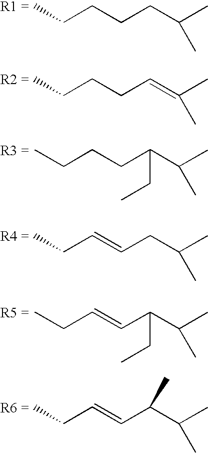

A represents a hydrogen atom or together with B a carbon-carbon bond
B represents a hydrogen atom, a hydroxy group or together with A a carbon-carbon bond,
C represents a hydrogen atom or together with D a carbon-carbon bond,
D represents a hydrogen atom or together with C a carbon-carbon bond,
E represents a hydrogen atom or together with F a carbon-carbon bond,
F represents a hydrogen atom or together with E a carbon-carbon bond,
or one of its esters, particularly of cholest-4-en-3-one oxime.

More precisely, the invention relates to a composition, particularly a pharmaceutical composition which can be administered orally, containing a compound as described previously and at least one oil chosen from sesame oil, olive oil, soya oil, cotton oil or a mixture of medium-chain triglycerides (such as for example ESTASAN®, MYGLIOL®), or a mixture of these different oils. Preferably, sesame oil is used.

The compounds of formula I, particularly cholest-4-en-3-one oxime such as for example those described in the international application WO 2004/082581, are generally highly insoluble in aqueous medium. With such active ingredients, difficulties appear during the development of chemically and physically stable formulations.

These compounds are potential cytoprotective compounds and can potentially be used as medicaments, particularly as medicaments which can be used in the treatment of neurodegenerative diseases.

It is therefore understood that a real need exists for a composition, particularly a pharmaceutical composition which can be administered orally, containing at least one compound of formula I, particularly cholest-4-en-3-one oxime, solubilized or in suspension, such that the compound is absorbed at the gastro-intestinal level.

The pharmaceutical composition according to the invention was developed according to the following criteria:
significant chemical stability over time of the compounds according to the invention;
highest possible concentration of active ingredient in solution/suspension;
simple oily solution, which is inexpensive in industrial production;
possible administration by an enteral probe;
taste which is acceptable or can be masked by standard flavourings;
possible administration to children or infants;
controlled viscosity allowing the filling of solid oral pharmaceutical forms.

By “pharmaceutical composition”, is meant in the present invention, a composition the components of which are pharmaceutically acceptable. For example, when oral administration is envisaged, the components are appropriate or acceptable for oral administration.

As the compounds of formula I, particularly cholest-4-en-3-one oxime are slightly soluble in water, a study was carried out by the Applicant in order to determine excipients which would allow them to be solubilized or put in suspension while satisfying the criteria listed above.

The Applicant discovered that the products described in this application exhibit very good behaviour with sesame oil, olive oil, soya oil, cotton oil or a mixture of medium-chain triglycerides (ESTASAN®, MYGLIOL® or equivalent) which unexpectedly produce much better results than in the presence of other oils tested, to the point of being able to prepare a sufficiently concentrated solution which allows a pharmaceutical preparation having the following advantages:
optimum chemical stability;
high concentration of solubilized active ingredient;
simple oily solution which is inexpensive in industrial production;
possible use for administration by an enteral probe;
acceptable taste;
ideal suitability for a paediatric form.

On the other hand these properties also allow the preparation of a solution or suspension which can be presented in the form of soft or hard gelatin capsules.

The content per capsule can be significant, which minimizes the number of capsules to be taken daily.

Optimum solubilization and suspension of the active ingredient have been obtained when the composition comprised as active ingredient at least one compound of formula I, particularly cholest-4-en-3-one oxime and an oil chosen from the following oils: sesame oil, olive oil, soya oil, cotton oil, the mixture of commercial medium-chain triglycerides ESTASAN®, MYGLIOL® or equivalent.

Thus, in its first subject, the invention relates to a composition, particularly a pharmaceutical composition, characterized in that it comprises at least:
+ a compound of formula I

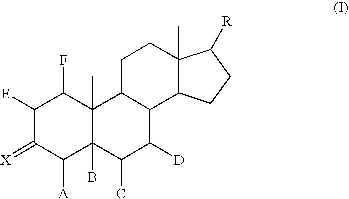

in which X represents an ═N—OH group,
R represents a group chosen from

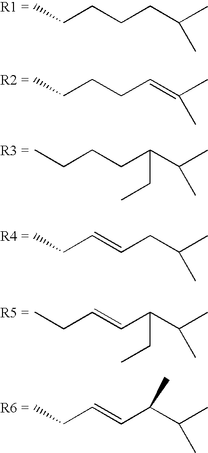

A represents a hydrogen atom or together with B a carbon-carbon bond
B represents a hydrogen atom, a hydroxy group or together with A a carbon-carbon bond,
C represents a hydrogen atom or together with D a carbon-carbon bond,
D represents a hydrogen atom or together with C a carbon-carbon bond,
E represents a hydrogen atom or together with F a carbon-carbon bond,
F represents a hydrogen atom or together with E a carbon-carbon bond,
or one of its esters, and
+ an oil chosen from sesame oil, olive oil, soya oil, cotton oil, or a mixture of medium-chain triglycerides (ESTASAN®, MYGLIOL® or equivalent) or a mixture of oils.

Preferably, the oil used is chosen from sesame oil, olive oil or soya oil, preferably sesame oil.

Preferably according to the invention, the compound of formula I can be chosen from cholest-4-en-3-one oxime, cholestan-3-one oxime, cholest-1,4-dien-3-one oxime, very preferably cholest-4-en-3-one oxime,
or one of their esters.

These compounds are described in the international application published on 30 Sep. 2004 under number WO 2004/082581.

According to the invention, the compound can be present in the pharmaceutical composition either in dissolved form, being able to be used in particular in enteral probes or in paediatric form; it is then a solution, either in the form of a suspension comprising solubilized active ingredient (saturating concentration) and active ingredient in solid form, making it possible to minimize the volume of the pharmaceutical form for a given dose, which is thus more acceptable for the patient.

In this composition, the compound is advantageously present in physiologically effective doses or representing a sub-multiple of the effective dose.

According to the invention, the compound can be present in the composition in a quantity ranging from 10 to 200 mg/ml in solution, preferably from 25 to 150 mg/ml), or in a quantity ranging from 30 to 500 mg/ml in suspension, preferably from 50 to 400 mg/ml.

The composition according to the present invention can be administered by oral route in an appropriate pharmaceutical form such as a hard gelatin capsule or a soft capsule or also a drinkable solution or a drinkable suspension of greater or lesser viscosity.

The composition according to the present invention can be used in mammals, more precisely in humans.

The compositions used by oral route can be presented in the form of gelatin capsules or other solid form which can comprise a lipid phase, drinkable solution or drinkable suspension, or ingestible form.

The quantities of the different constituents of the compositions according to the invention are those used in a standard fashion in the fields considered.

In a known manner, the composition of the invention can also contain adjuvants which are usual in the fields considered, such as for example preservatives, antioxidants, pigments and dyestuffs, thickeners, surfactants, flavourings, sweeteners, the agents stabilizing the active ingredient particles.

The quantities of these different adjuvants are those used in a standard fashion in the fields considered, and for example from 0.0001% to 10% of the total weight of the composition. These adjuvants are introduced into the oil phase.

The following examples illustrate the invention without limiting it.

### EXAMPLE 1

### Comparison of the Stability of Cholest-4-en-3-one Oxime in Different Oils

The stability of cholest-4-en-3-one oxime was tested in 12 different oils. The compound is added to the oil at a concentration of 15 mg/ml in the form of micronized powder, and the mixture is placed in an oven at 60° C. for 15 days. The quantity of impurities is measured in the suspension which is diluted so as to exhibit a concentration of compounds at 2.5×10⁻⁴M, by HPLC UV at 255 nm. The major impurity obtained is cholestenone. Other impurities can also appear, certain of which are known (and known to be stable), whereas other impurities appearing are unknown, and the potential toxicity or stability of which is therefore not known.

The stabilities are shown in the table below. The product is considered to be very stable (++, very stable) in a given oil if after the incubation for 15 days in the oven at 60° C., the percentage of the measured total area under the curve of the chromatogram obtained corresponding to peaks of degradation products of cholest-4-en-3-one oxime represents less than 1%, and these are known, stable products. The product is considered stable (+, stable) in an oil if after the 15 days spent in the oven at 60° C., the percentage of the measured total area under the curve of the chromatogram obtained corresponding to peaks of degradation products of cholest-4-en-3-one oxime represents less than 1%, but corresponds to unknown degradation products. The product is considered unstable (−, instable) in an oil if after the 15 days spent in the oven at 60° C., the percentage of the measured total area under the curve of the chromatogram obtained corresponding to peaks of degradation products of cholest-4-en-3-one oxime represents more than 1%.

| | stability |
|---|---|
| Sesame oil | ++ |
| Olive oil | ++ |
| Soya oil | ++ |
| Cotton oil | ++ |
| Mixture of medium-chain triglycerides | ++ |
| (ESTASAN ®, MYGLIOL ®) | |
| Caster oil | + |
| Ground nut oil | + |
| Rapeseed oil | + |
| Corn oil | − |
| Almond oil | − |
| Sunflower oil | − |
| Oleic acid | − |

(−): major instability of the product in the oil tested, with release of more than 1% degradation products, over 2 weeks at 60° C.
(+): compound which is stable in the oil tested, with release of less than 1% unknown degradation products, over 2 weeks at 60° C.
(++): compound which is very stable in the oil tested, with release of less than 1% stable known degradation products, over 2 weeks at 60° C.

### CONCLUSION

The preferred oils are those which least degrade cholest-4-en-3-one oxime in the mixtures over time. These are sesame oil, olive oil, soya oil, cotton oil, the mixture of medium-chain triglycerides (ESTASAN®, MYGLIOL®), castor oil, ground nut oil, and rapeseed oil. Corn oil, almond oil, sunflower oil and oleic acid produce unsatisfactory results. Among the oils with which the least degradation products are obtained, those degrading to identified compounds and known to be stable over time (results not shown) are preferred. In this classification, the best oils are sesame oil, olive oil, soya oil, cotton oil and the mixture of medium-chain triglycerides (ESTASAN®, MYGLIOL®). Among the latter, sesame oil, olive oil and soya oil are preferred, as they have the most acceptable taste among all of the 5 preferred oils. Sesame oil is the preferred oil among these three oils as it allows the highest stability of cholest-4-en-3-one oxime.

### EXAMPLE 2

### Comparison of the Solubility of Cholest-4-en-3-one Oxime in Water and in Different Oils

Tests were carried out in order to demonstrate the maximum solubility of cholest-4-en-3-one oxime in water and in the different oils retained following the stability test (Example 1).

| | maximum solubility |
|---|---|
| | (mg/ml) |
| Water | 0.001 |
| Sesame oil | 35 |
| Olive oil | 40 |
| Soya | 30 |
| Cotton | 28 |
| Mixture of medium-chain triglycerides | 45 |
| (ESTASAN ®, MYGLIOL ®) | |
| Castor | <15 |
| Ground nut | <15 |
| Rapeseed | <15 |

The best solubility was obtained with the mixture of medium-chain triglycerides (ESTASAN®, MYGLIOL®), followed by olive oil, sesame oil and soya oil.

### EXAMPLE 3

### The Following Composition is Produced

| Component: | % by weight |
|---|---|
| Cholest-4-en-3-one oxime | 43.4 |
| Refined sesame oil* | 50.8 |
| Surfactant adjuvant: soya lecithin* | 1.0 |
| Thickening adjuvant: mixture of hydrogenated | 48 |
| vegetable oils, NF type II** | |

*according to European Pharmacopoeia standards
**according to US Pharmacopoeia standards

The composition is encapsulated, preferably in soft capsules, and is therefore presented in solid form. The advantage of this composition is the significant quantity of compound put in suspension per capsule, which limits the volume taken. There is no need to flavour the mixture before encapsulation, since the mixture has an acceptable taste.

Capsules containing this composition (165 mg of cholest-4-en-3-one oxime per capsule) were stored in plastic bottles closed with a non-hermetic plastic stopper (50 capsules per bottle) at 40° C. and 75% humidity for 1 and 2 months. After 1 and 2 months, the composition is stable since the level of impurities which has recently appeared is less than 1% in total.

### EXAMPLE 4

### The Following Composition is Produced

The compound cholest-4-en-3-one oxime is dissolved at 30 mg/ml in sesame oil. The composition thus obtained is presented in the form of a solution.

This liquid composition is particularly useful as it is generally easier to swallow than a capsule, the dose can be easily adapted by pipetting the necessary volume. The mixture requires neither flavouring, nor the addition of sweetener, as the taste is acceptable. This composition has the advantage of being simple and inexpensive. Furthermore, it corresponds to the pharmaceutical criteria described for a paediatric form and can therefore be used in these populations or for older populations who can only be fed by enteral probe.

The composition is particularly stable after at least 2 weeks at 40° C. since the level of impurities which has recently appeared is less than 1% in total. It is also stable at 60° C.

1. Composition, particularly pharmaceutical composition, characterized in that it comprises at least:
+ a compound of formula I 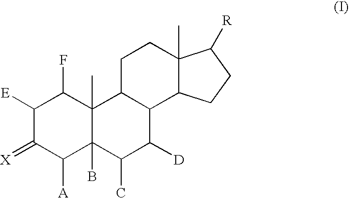
in which X represents an ═N—OH group,
R represents a group chosen from 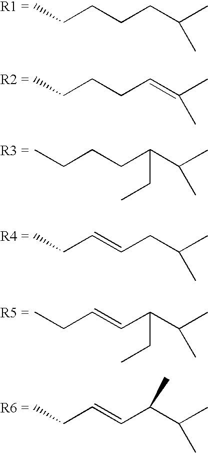
A represents a hydrogen atom or together with B a carbon-carbon bond
B represents a hydrogen atom, a hydroxy group or together with A a carbon-carbon bond,
C represents a hydrogen atom or together with D a carbon-carbon bond,
D represents a hydrogen atom or together with C a carbon-carbon bond,
E represents a hydrogen atom or together with F a carbon-carbon bond,
F represents a hydrogen atom or together with E a carbon-carbon bond,
or one of its esters, and
+ an oil chosen from sesame oil, olive oil, soya oil, cotton oil, or a mixture of medium-chain triglycerides (ESTASAN®, MYGLIOL®) or a mixture of oils, preferably sesame oil, olive oil and soya oil, still more preferably sesame oil.

2. Composition according to claim 1, characterized in that the compound of formula I is chosen from cholest-4-en-3-one oxime, cholestan-3-one oxime, cholest-1,4-dien-3-one oxime, very preferably cholest-4-en-3-one oxime, or one of their esters.

3. Composition according to one of claim 1 or 2, characterized in that the compound is present in the composition in the form of solution in a quantity ranging from 10 to 200 mg/ml.

4. Composition according to one of claim 1 or 2, characterized in that the compound is present in the composition in the form of suspension in a quantity ranging from 30 to 500 mg/ml.

5. Composition comprising the compound cholest-4-en-3-one oxime and sesame oil.

6. Composition comprising the compound cholest-4-en-3-one oxime, sesame oil, soya lecithin and a mixture of hydrogenated vegetable oils.
